(19) United States
(12) Patent Application Publication
(10) Pub. No.: US 2004/0043417 A1
(43) Pub. Date: Mar. 4, 2004

(54) Generation and screening of a dynamic combinatorial library

(76) Inventors: Jean-Marie Lehn, Strasbourg (FR); Olof Ramstrom, Strasbourg (FR)

Correspondence Address: BURNS DOANE SWECKER & MATHIS L L P, POST OFFICE BOX 1404, ALEXANDRIA, VA, 22313-1404, US

(21) Appl. No.: 10/220,470

(22) PCT Filed: Mar. 1, 2001
(86) PCT No.: PCT/EP01/02310

(30) Foreign Application Priority Data
Mar. 1, 2000 (EP) 00104236.5

(51) Int. Cl. G01N 33/53; G01N 33/543

(52) U.S. Cl. 435/7.1; 436/518

(57) ABSTRACT

The present invention concerns a method for selectively establishing a dynamic combinatorial library of ligands for a target which binds at least two functionalities, which method comprises the following steps: selecting a plurality of functionalities which upon combination with each other ire capable of forming an entity which may bind to the at least two functionalities in the target; selecting at least one spacer group to be located between the at least two functionalities, which spacer group is of an appropriate size and/or flexibility to allow the functionalities to fit into the binding sites on the target and allowing a reversible bond formation and cleavage; creating discrete ligands by linking at least two identical or different functionalities by at least one spacer group or; linking the functionalities to fragments of the spacer group which contain functions allowing the said reversible bond formation under formation of the spacer group and cleavage of it; mixing together a plurality of different discrete ligands and/or different functionalities; subjecting the mixture to conditions allowing a reversible bond formation and cleavage; adding the target to the mixture; identifying the functionality combinations which are most appropriate for the formation of a complex between the target and the active molecule. In a further embodiment of the invention, the target is added when the discrete ligands are mixed together, in order to be present when the scrambling takes place.

### TECHNICAL FIELD AND PRIOR ART

The present invention relates to a method for selectively producing a dynamic combinatorial library (DCL) for binding to a target. More specifically, the dynamic combinatorial library which is established with the method according to the present invention is directed to molecules which contain at least two functionalities which are capable of binding to the target. These functionalities are linked by a dynamic spacer group allowing an interchange of the functional groups around the spacer group.

New chemical or biological entities with useful properties are generated by identifying a chemical or biological compound (a so-called lead compound) with some desirable properties or activities, creating varieties of said compound to form a library, and evaluating the properties and activities of those variant compounds.

The drawback of that method is that only a small sub-set of the library is usually accessible for biological tests.

Thus, the conventional approach is limited by the relatively small pool of previously identified compounds which may be screened to identify new compounds with the desirable property or activity.

Another drawback pertains to the step of the creation of variants. Traditionally, compound variants are generated by chemists or biologists using a conventional chemical or biological synthesis procedure. Thus, the generation of compound variants is time-consuming and requires huge amounts of work.

Recently, attention has focused on the use of combinatorial chemical libraries to assist in the generation of new chemical compounds.

Combinatorial chemistry has experienced an explosive growth in recent years, as it provides a powerful methology for exploring the molecular geometrical and interactional spaces through molecular diversity generation. This is in particular the case for the discovery of new biologically active substances and medical drugs. It resides on the constitution of vast combinatorial libraries (CLs), extensive collections of molecules derived from a set of units connected by successive and repetetive application of specific chemical reactions. It is thus based on a large population of different molecules that are present as discrete entities.

In the spirit of Emil Fischer's “Lock and Key” metaphor the constitution of a CL of substrates amounts to the fabrication of a large collection of keys, with the goal that one of them will fit the target lock/receptor and be retrievable from the mixture.

In contrast to this, the present invention makes use of a different concept to establish combinatorial libraries, namely of the so-called dynamic combinatorial chemistry which is a conceptionally different approach that rests on supra-molecular chemistry. It relies on a reversible connection process for the spontaneous and continuous generation of all possible combinations of a set of basic components, thus making virtually available all structural and interactional features that these combinations may present. Such multicomponent self-assembly amounts to the presentation of a dynamic combinatorial library (DCL) which is a potential library made up of all possible combinations in number and nature of the available components. This is followed by selection of the component among all those possible, that possesses the features most suitable for formation of the optimal supramolecular entity with the target site, by recruiting the correct partners from the set of those available. The degree of completeness of the set of components/subunits depends on the extent to which the possible combinations cover the geometrical and interactional spaces of the targets.

Self-assembly in a multi-component system is a combinatorial process with a search procedure directed by the kinetic and thermodynamic parameters imposed by the nature of components and their interactions.

In WO 97/43232 there is disclosed a substance library which library consists of molecular species which are bonded to a molecular pairing system. The pairing system is composed of molecules, in the preferred embodiment, which are selected amongst specially designed nucleic acids which can bind to each other in a certain manner which results in a particular geometric form. The molecular species are selected, in a preferred embodiment, from the group consisting of peptides, and these peptides are designed according to the particular requirements of a given substrate which is brought into contact with the library component. The complex which forms upon contact with the substrate is identified in order to evaluate the interaction between a substrate and complex.

In Proc. Natl. Acad. Sci. USA 1997 (94), 2106, Ivan Huc and Jean-Marie Lehn disclose a method for the generation of a dynamic combinatorial library of imines from structural fragments bearing aldehyde and amino groups. The method is directed toward the synthesis of inhibitors of the enzyme carbonic anhydrase by recognition-involved assembly, and the synthesis of the above-mentioned imines is carried out in the presence of the said enzyme carbonic anhydrase. It was found that reversible combination of the used amines and aldehydes leads to the shift of the equilibrium population towards the imine product that was closest in structure to a known highly efficient inhibitor of the enzyme.

### DETAILED DESCRIPTION OF THE INVENTION

The problem underlying the present invention is to find a method for creating a dynamic combinatorial library which library is composed of molecules which contain at least two discrete functionalities capable of binding to the target and which method allows an interchange of the functionalities in order to create combinations of different functionalities which interact with the receptor in different ways.

This object is attained by a method for selectively establishing a dynamic combinatorial library of ligands for a target which binds at least two functionalities, which method comprises the following steps:

(i) selecting a plurality of functionalities which upon combination with each other are capable of forming an entity which may bind to the at least two functionalities in the target;

(ii) selecting at least one spacer group to be located between the at least two functionalities, which spacer group is of an appropriate size and/or flexibility to allow the functionalities to fit into the binding sites on the target and allows a reversible bond formation and cleavage;

(iiia) creating discrete ligands by linking at least two identical or different functionalities by at least one spacer group; or

(iiib) linking the functionalities to fragments of the spacer group which contain functions allowing the said reversible bond formation under formation of the spacer group and cleavage of it;

(iv) mixing together a plurality of different discrete ligands and/or different functionalities;

(v) subjecting the mixture to conditions allowing a reversible bond formation and cleavage;

(vi) adding the target to the mixture;

(vii) identifying the functionality combinations which are most appropriate for the formation of a complex between the target and the active molecule.

This object is also attained by a method for selectively establishing a dynamic combinatorial library of ligands for a target which binds at least two functionalities, which method comprises the following steps:

(I) selecting a plurality of functionalities which upon combination with each other are capable of forming an entity which may bind to the at least two functionalities in the target;

(II) selecting at least one spacer group to be located between the at least two functionalities, which spacer group is of an appropriate size and/or flexibility to allow the functionalities to fit into the binding sites on the target and allowing a reversible bond formation and cleavage;

(IIIA) creating discrete ligands by linking at least two identical or different functionalities by at least one spacer group; or

(IIIB) linking the functionalities to fragments of the spacer group which contain functions allowing the said reversible bond formation under formation of the spacer group and cleavage of it;

(IV) mixing together a plurality of different discrete ligands and/or different functionalities in the presence of the target;

(V) subjecting the mixture to conditions allowing a reversible bond formation and cleavage, hence a scrambling of the functionalities;

(VI) identifying the functionality combinations which are most appropriate for the formation of a complex between the target and the active molecule.

In one embodiment of the methods described above, the final mixture is analysed and the result compared to the result obtained on a mixture obtained under identical conditions, but without the addition of the target.

It was found that it is possible to generate dynamic combinatorial libraries (DCL's) by linking functionalities with a dynamic spacer group in which reversible covalent bonds can easily and reversibly be formed and broken, hence allowing for an interchange reaction along or inside the said spacer group.

The method may thus be useful for determining which part of the molecule is responsible for the affinity of the target.

Thus, the ligands that are assayed are fragmented in basic components by splitting a reversible bond between at least two parts of the molecule under conditions allowing such a splitting. The ligands are formed by the reversible generation of the bond located along or inside the spacer group.

By allowing the basic components to reassemble and compete for the target, it is thus possible to determine which compound or moiety has an affinity for the target and eventually which compound or moiety has the best affinity for the target.

The concept of the invention is based on an approach that rests on supramolecular chemistry.

The method according to the present invention is in particular appropriate for the generation of libraries of molecules for target substrates in which one active binding site is specific for two or more functionalities. This is a preferred embodiment of the present invention. However, the method is also appropriate for the generation of libraries of molecules which bind to two or more different binding sites in a given target.

When in the context of the present invention there is referred to “functionality”, this means any polar, nonpolar, hydrophilic or lipophilic, or charged unit or subunit or electron donor or electron acceptor group. “Functionality” on the one hand includes simple functional groups like amino and imino groups and derivatives thereof, hydroxy and mercapto groups and derivatives thereof, oxo and thioxo groups, formyl and thioformyl groups, aryl groups, substituted aryl groups, phenyl groups, substituted phenyl groups, pyridyl groups and derivatives thereof, carboxy groups and carboxylato groups and derivatives thereof, alkyloxycarbonyl groups. (di)thiocarboxy groups and derivatives thereof, (di)thiocarboxylato groups, carbamoyl groups and derivatives thereof sulfo, sulfino and sulfeno groups and derivatives thereof, alkyloxysulfonyl, alkyloxysulfinyl and alkyloxysulfenyl groups, sulfamoyl, sulfinamoyl and sulfenamoyl groups and derivatives thereof, cyano and (iso)(thio)cyanato groups, hydroperoxy groups, nitroso groups, hydroxyamino groups, hydrazino groups, —NR₁R₂, —⁺NHR₁R₂ and —⁺NR₁R₂R₃ groups, wherein R₁, R₂ and R₃ are identical or different and represent alkyl, cycloalkyl, alkylcycloalkyl, aryl, alkylaryl with 1 to 40 C atoms, —⁺OR₁R₂ groups wherein R₁ and R₂ are identical or different and represent alkyl, cycloalkyl, alkylcycloalkyl, aryl, alkylaryl with 1 to 40 C atoms, hydrazide groups and any other suitable groups known to a person skilled in the art.

On the other hand, “functionality” also includes more complex components which themselves may contain functional groups, and non-limiting examples include heterocycles carrying one or more heteroatoms in the ring selected from the group consisting of N, O and S, amino acids and oligo- and polypeptides, sugars (preferably hexoses and pentoses), sugar derivatives (like peracetylated sugars) and oligomers and polymers thereof, and nucleic acids and derivatives thereof.

According to the invention, the terms used herein have the following meanings:

“ligand” preferably means a chemical or biological molecule with a molecular weight typically not greater than 1000, preferably not greater than 500, advantageously not greater than 200, which possesses an affinity for a target, i.e. that is able to interact with the target by forming simultaneaously a plurality of weak bonds such as hydrogen bonds, hydrophobic interactions etc.

The scope of the present invention is not limited to biological targets. Any natural of synthetical organic and inorganic target may be used. In general, any kind of target for which an activity assay exists, is suitable. The activity may e.g. be determined, by measuring the change of fluorescence, viscosity, conductivity or IR or UV absorption. Therefore other suitable targets may be zeolithes, clathrates, oligonucleotides, oligopeptide, oligosaccharides, sensors, clusters. RNA aptamers, organic and inorganic catalysts, ionophores, any kind of macrocycles like metallomacrocycles, macrocyclic lactams, macrocyclic esters, macrobicyclic cryptands and macrocyclic oligocholates; any kind of synthetic polymers like polyaminoacids, polyamides, polyesters, polyalcohols and mixtures thereof, etc. Ligands may have any kind of functional groups mentioned above. Even simple “molecules” like cations or anions may act as ligands.

In general, all kinds of molecules, the one of which can act as a ligand and the other one as a target, are suitable to be used in the method according to the present invention.

“target” means a biological macromolecule with a molecular weight typically greater than 500, preferably greater than 1000, which is of proteinaceous nature, including lipoproteins, glycoproteins and analogs of proteins, wherein either the peptide bond CO—NH— is replaced by an analogous bond, eventually reversible such as an imine, ester, sulfonamide, sulfone, sulfoxide, phosphate, phosphonate, phosphonamide, guanidine, urea, thiourea, or imide bond, or wherein the aminoacids are replaced with aminoacids different from the 20 aminoacids found in natural proteins. The natural proteins may have different functions. The may act as enzymes, as receptors, or as antibodies. Receptors may, for example, be membrane receptors, hormone receptors, or signal transducers.

“reversible bond” means that the half time of the bond formation or cleavage is typically less than one month, preferably less than one day and more preferably less than one hour.

An important point in the present invention is the spacer group located between the functionalities which must comprise in its chain atoms or functional groups which allow a reversible formation and cleavage of bonds and hence a scrambling process resulting in an interchange of functionalities. Those chain atoms or functional groups can be the spacer group itself. The chain atoms or functional groups can also be located at the ends of an entity forming the backbone of the spacer group and linking the functionalities to the said backbone, thus creating the spacer group. The chains or functionalities can also be located in the middle of the spacer group or at an appropriate position within the spacer group. There can be one or more chain atoms or functional groups allowing a reversible bond formation and cleavage. In case more than one of these groups or atoms are present, they can be identical or different.

Further prerequisites an appropriate spacer group has to fulfill are, besides the reversibility of the bond formation, an easy controllability and the possibility to be operated under mild conditions. Furthermore., the compatibility with the binding of the functionality to the target is an important feature for an appropriate spacer group for a given target. This means that the spacer group must have an appropriate size and/or an appropriate flexibility to create a conformation which allows the molecules and more particularly the functionalities to fit into the binding site. One method to establish the appropriate size of the spacer group is, for example, analyzing the size of the binding site by appropriate methods like, for example, an X-ray structural analysis. Another method is, for example, to evaluate the size of a known ligand for a given binding site. If such known ligand is a ligand wherein a spacer group is already present between the functionalities which bind to the target, the size of the spacer group will be taken as a guideline to create an appropriate spacer group. It was found out that it is not necessary to mimic exactly the original size of a “natural” spacer group in an “artificial” spacer group as long as the “artificial” spacer group is sufficiently flexible, to adapt a conformation allowing the functionalities present to fit into the binding site on the target.

Reversible covalent reactions are especially suited, since bond formation and cleavage may occur in particular conditions, and may be inhibited in others. It is preferred if the reaction/connection can take place in or near physiological conditions. In general, the amount of the thermodynamically most stable supramolecular species will be the largest amount. Examples of reversible covalent reactions are those where carbonyl groups are involved like the formation of imines and analogues like hydrazones, acylhydrazones, sernicarbazones and analogues thereof, amides, acetals, and esters, alcohol exchange in borate esters, reversible Diels-Alder and other thermal- or photoinduced rearrangements like sigmatropic and electrocyclic rearrangements, and Michael reactions or alkene or alkine metathesis using catalysts that may be soluble in water are further examples. Another example are photoinduced interconversions. The reaction of amino groups with carbonyl groups to imines, oximes, bydrazones, acyhydrozones or semicarbazones is a preferred reaction. A further preferred reaction is the thiol exchange in disulphides.

Preferably, the spacer group allows to lock the process by a simple reaction. When designing a dynamic library making use of the method to the present invention, two different procedures can be employed. In one procedure, library generating and screening is performed in one single step; in the other procedure, these two steps are carried out separately. The particulars for the above-mentioned different procedures are the following:

a) adaptive, combinatorial libraries (one-step procedure): the generation of the library constituents is conducted in a single step in the presence of the target, so that the library composition may adjust leading to selection and amplification of the preferred substrate(s); the DCL may be real or virtual; screening by the target occurs in parallel with the reversible generation of the library constituents; this is the approach where the dynamic features are operative over the whole process;

b) pre-equilibrated dynamic combinatorial libraries (PDCL) (two-step procedure): the constituents of the library are generated by reversible interconversion and equilibration in absence of the target, which is added in a second step after reversibility has been stopped; this has the advantage that one may use reversible reactions which are not compatible with the presence of the target but the process is not adaptive and no amplification of the preferred substrate can result; it is however sufficient for lead generation, i.e. the discovery of species presenting the activity sought for; in its second phase it amounts to the usual, static combinatorial chemistry approach where an actual, real library is screened by the target; the resulting library may be termed pre-equilibrated or postdynamic combinatorial library (pDCL).

Generally, the target is a natural protein, namely an enzyme, a receptor or an antibody. Receptors may be membrane receptors, hormone receptors, signal transducers, etc.

When the target is an enzyme, the ligand that is sought to be obtained may act as a substrate, an inhibitor or an activator for said enzyme.

When the target is a receptor, the partner that is sought to be obtained may act as a natural or artificial ligand, an agonist or an antagonist for said receptor.

When the target is an antibody, the partner that is sought to obtain will act as an antigen for said antibody.

The basic components are selected according to their chemical structure.

In a more preferred embodiment, the method according to the present invention is used to design libraries of molecules in which the two or more functionalities are selected from the group consisting of carbohydrates and sugars, for example pentoses and hexoses which may be chemically modified, if desired. This is important in so far as carbohydrate recognition plays an important role in many biological processes, such as cell-cell interactions and cell communication. In addition, a multitude of enzymes are involved in various carbohydrate mediated processes associated with, e.g., defence, cell proliferation and cell death, as well as in general carbohydrate metabolism. Carbohydrates and, respectively, sugars are highly attractive tools for generating mimics and analogues of such recognition processes. Possible applications of the binding of molecules containing two or more carbohydrate or sugar functionalities to a target are, for example, the direct or indirect inhibition of carbohydrate-recognizing proteins, e.g. enzymes, or the finding of potential agonists/antagonists of carbohydrate receptors. The method according to the present invention allows to design libraries of compounds which are capable of being applied in those fields.

In an even more preferred embodiment, two or more carbohydrates or sugars, preferably from the group consisting of optionally modified pentoses and hexoses, are linked by a spacer group with a central disulfide bridge which is symmetrically substituted by a —(CH₂)ₙ group with n being an integer from 1 to 5, preferably 2 or 3. Those —(CH₂)ₙ-groups are then linked to the sugar of carbohydrate functionalities by a —C(O)—(C₆H₄)—O-group. The disulfide bridge is formed from two thiol units. The process according to the present invention can be carried out % vith the ligand in which the functionalities are linked by the spacer group. The process can also be carried out starting from the thiol units containing the functionalities, i.e. the sugars.

According to a variant of the even more preferred embodiment, the two or more carbohydrates or sugars are linked by a spacer group which contains one or more acylhydrazones and/or imines. The acylhydrazones are formed from hydrazides and aldehydes, the imines are formed from amines and aldehydes. The hydrazides, aldehydes and imines are linked to the sugar by an ether group formed between a hydroxyl function in the sugar and the organic rest to which the amino-, aldehyd- or hydrazide-function is bound. The organic rest can be an aliphatic or aromatic rest. The acylhydrazones and imines can be formed in a reaction between aldehydes and hydrazides and amines, respectively, which are each linked to the sugar in the above-specified way. It is also possible to add a further difunctional unit which contains aldehyde-, hydrazide and/or amino functions at its end. This difunctional unit then reacts with the respective function linked to the sugar. In this way, a spacer group containing two reversible bonds can be formed.

In the most preferred embodiment of the present invention, the process is used to establish a DCL for the binding of carbohydrates to those carbohydrate-binding proteins belonging to the class of lectins and toxins. Examples for lectins are S-type lectins (galectin), C-type lectins (selectin), or P-type lectins. Examples for toxins are Shiga and Shiga-like toxins.

As an example for lectins, Concanavalin A (Con A) was chosen which is specific for a branched trimannoside core unit, located in N-glycosidic carbohydrate-peptide linkages of glycoproteins often associated to cell surfaces. Due to this Con A is extensively used as a tool in histochemical staining.

The binding site of Con A is rather shallow, composed of a number of threonine and tyrosine hydroxyl, aspartate carboxyl, as well as asparagine and main chain amide groups, capable of forming a network of hydrogen bonds to the natural substrate, the branched trimannoside unit (Man)₃. The majority of these interactions are formed with the non-reducing, peripheral mannosides of (Man)₃, rather than with the central mannose group, thus implying that the latter acts more or less as a linker between the interacting parts.

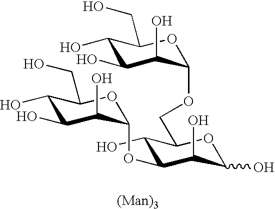

It was possible to mimic this interaction by a structure containing two carbohydrate head groups joined by a spacer containing one or two reversible covalent bonds as will be described in the following.

The design of the initial carbohydrate library was based on considerations concerning the mimicking of the native trimannoside unit. A size of the spacer as close as possible to the situation in trimannoside is one option. However, a slightly longer, but sufficiently flexible chain, can be used as well. Such a compound would then be able to fold into a conformation suitable for fitting into the site. Obviously, an excessively extended linker would result in a lower binding, because of a higher entropical loss upon binding.

### EXAMPLE 1

The compounds 1-6 listed in Table 1 were chosen a library of carbohydrate homodimers, containing a disulphide bridge in the spacer. A phenylamino group was also introduced, primarily because it provided a chromophore for the subsequent HPLC-analysis. Three hexopyranosides (D-mannose, D-glucose, D-galactose), and two pentopyranosides (L-arabinose, D-xylose) were used as carbohydrate head groups, and two different spacers, varying in length by one methylene group were examined.

TABLE 1
Structures of the disulphide linked carbohydrate dimers 1-6

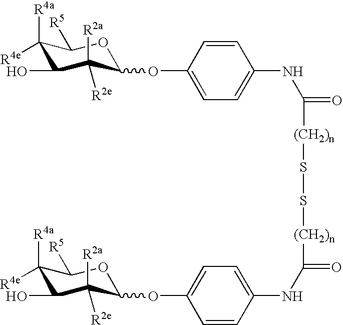

| Compound | α/β | R^{2a} | R^{2c} | R^{4a} | R^{4c} | R⁵ | n |
|---|---|---|---|---|---|---|---|
| 1 (D-Man) | α | OH | H | H | OH | CH₂OH | 3 |
| 2 (D-Gal) | β | H | OH | OH | H | CH₂OH | 2 |
| 3 (D-Gal) | β | H | OH | OH | H | CH₂OH | 3 |
| 4 (D-Glc) | β | H | OH | H | OH | CH₂OH | 2 |
| 5 (L-Ara) | β | H | OH | OH | H | H | 2 |
| 6 (D-Xyl) | β | H | OH | H | OH | H | 2 |

The libraries resulting from scrambling of the original dimers were generated by mixing the dimers 1-6 together with an initiating reagent, capable of reducing some of the disulphides to the corresponding thiols. Dithiothreitol (DTT) was found to be a good choice, since this compound oxidises efficiently to a stable 6-membered cyclic disulphide that may be expected not to take part in the scrambling of the library disulphides. Upon initiation/reduction, interconversion between the disulphides occurred, the rate of which was highly dependent upon the pH of the solution. At high pH (>8), scrambling was achieved reasonably rapidly.(within hours), whereas at low pH (<5) no scrambling could be detected. On the other hand, without initiation, no significant scrambling occurred within two weeks. A similar pH-dependence was recorded for the binding of the carbohydrates to the receptor, where a pH close to neutral was preferable. A pH of 7.4 was chosen as a level at which a reasonable rate of scrambling could be obtained, while receptor binding was not significantly affected. Alternatively, the library can also be generated starting from the thiols.

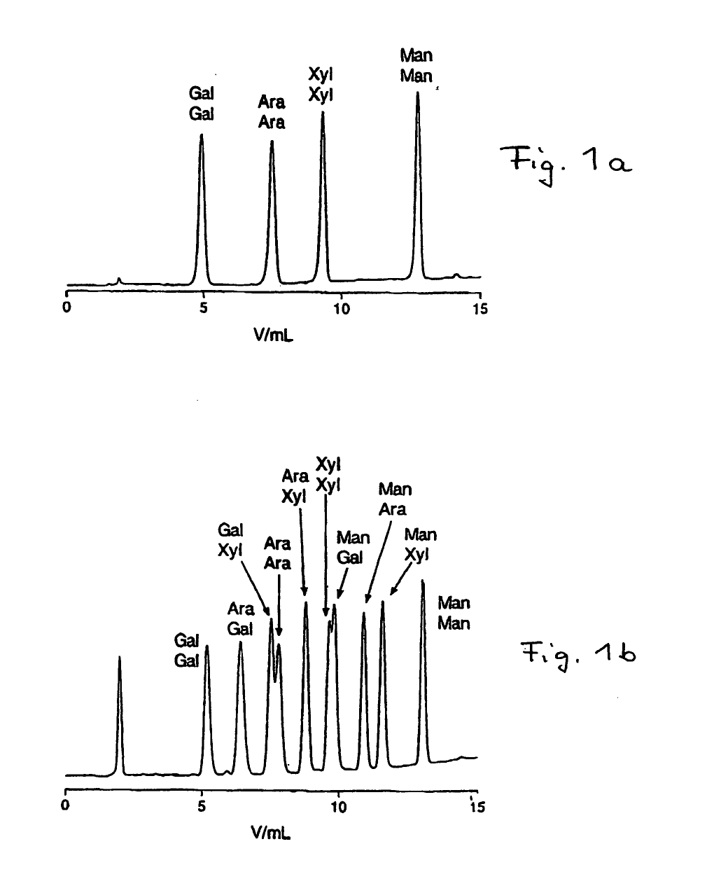

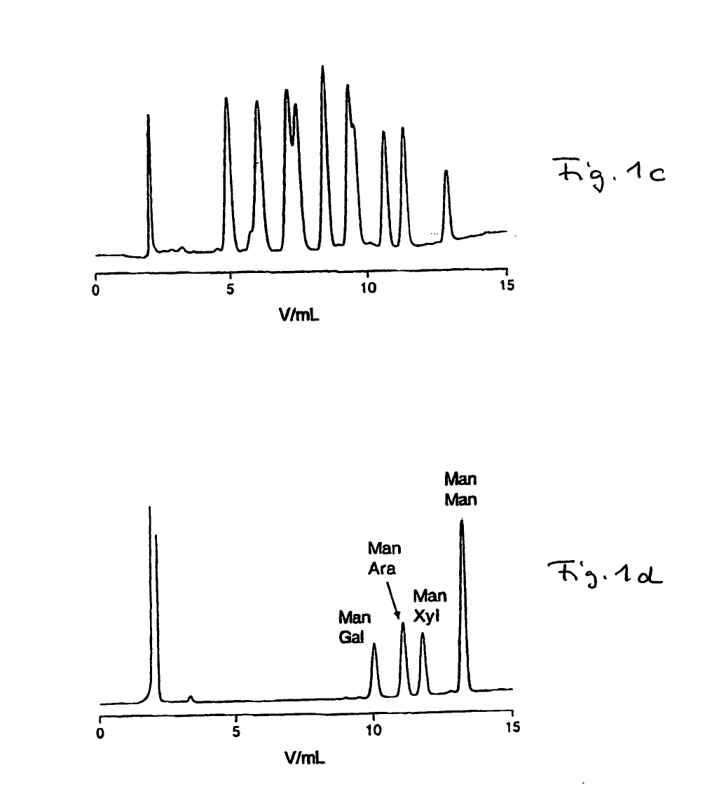

In the case of the library of size 10 (n=4), in the absence of any receptor, scrambling occurred smoothly at pH=7.4 from the original species (FIG. 1a) generating all ten expected ditopic combinations in comparable amounts as analyzed by RB-HPLC (FIG. 1b). When the receptor in the form of sepharose-bound Con A was added to the equilibrating pool, a shift in the concentrations of the different constituents present unbound in solution was recorded (FIG. 1c) The fraction of some of the free species decreased, notably the D-mannose-containing homo- and heterodimers. In order to characterize the species actually bound to the receptor, the Con A-sepharose beads were eluted by acidifying the solution to pH 4 and the composition of the eluate was analyzed (FIG. 1d). The assignment of the peaks to the different constituents of the scrambled libraries was made on the basis of the relative retention times. Clearly, the D-mannose homodimer was most efficiently bound to the lectin, and to a lesser extent the D-mannose containing heterodimers. All other species in the equilibrating pool did not bind to Con A-sepharose and remained in solution. Thus, the receptor could be used to “fish out” the best bound species from the equilibrating pool, while leaving the others behind.

When the receptor was present during the entire scrambling process, a slightly higher yield of the bis-mannoside could be recovered, compared to when it was added upon pre-equilibration. In the former case, the amount of bis-mannoside was approximately 2.1 times higher than each of the D-mannose containing heterodimers. In the latter situation, this ratio decreased to 1.5 times. This result is indicative of an adaptive effect where the lectin to some extent acts as a thermodynamic trap during scrambling.

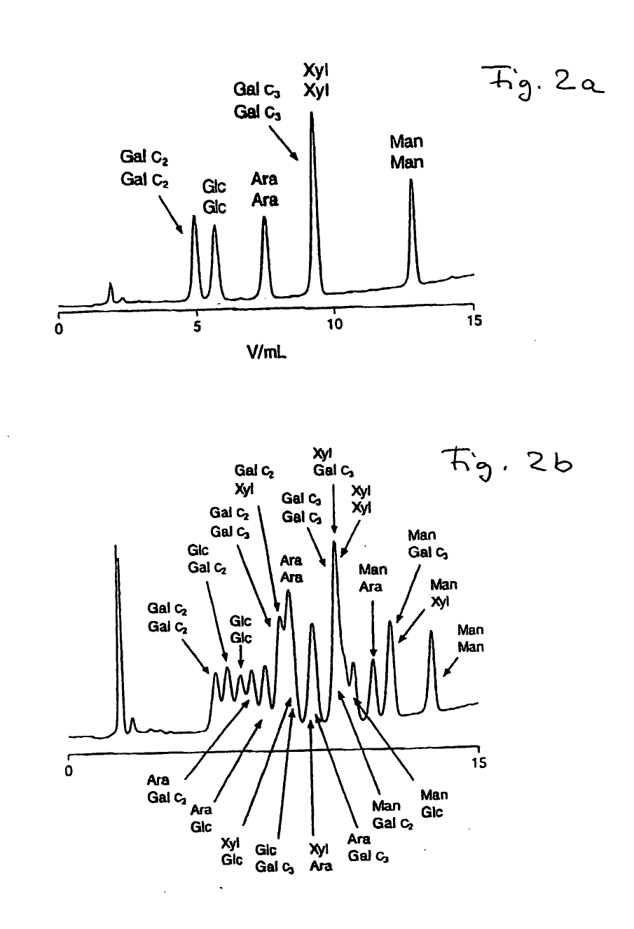

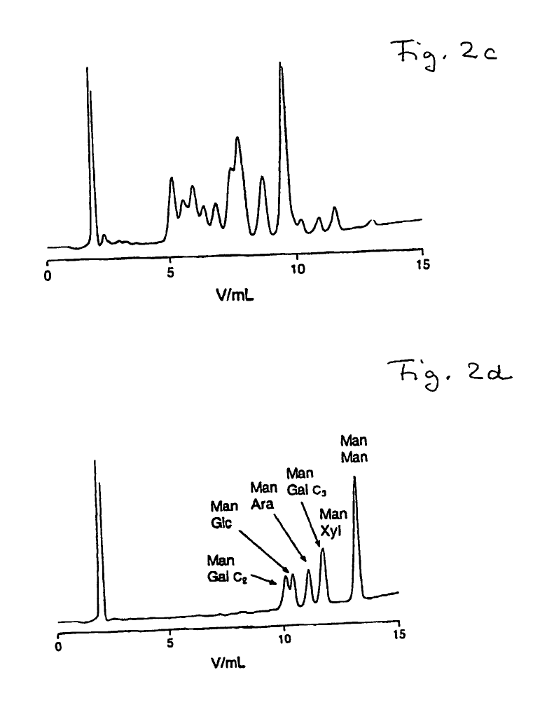

The same behavior, albeit less pronounced was observed for the 21-library (n=6. FIG. 2). Due to the larger number of species resulting from the scrambling, difficulties in their separation were encountered with the system used, and not all components could be resolved in the HPLC conditions used (FIGS. 2a, 2b). This was particularly the case for the homodimer containing D-galactose and a linker with 3 methylene units (Gal C3), and the D-xylose homodimer (Xyl), which could not be distinguished by reverse phase chromatography (FIG. 2a). However, from cross-reference studies the resulting chromatographic separation pattern was as could be expected, similar to the resulting 10-library. Upon elution of the Con A-sepharose and analysis of the eluate (FIG. 2d), a much clearer picture could be attained: mainly the D-mannose homodimer and to some extent the D-mannose containing heterodimers were found to be bound, while all other library constituents remained in the eluate.

### EXAMPLE 2

In addition to using disulphide interchange processes in generating dynamic combinatorial carbohydrate libraries, acyl exchange processes can be used. In this example, acyl hydrazone and imine interchange processes were examined. The reversible formation of acyl hydrazones is advantageous for generating DCLs as the formation and component interchange processes are rapid in acidic aqueous condition and rather slow in neutral and basic conditions. The acyl hydrazones formed are sufficiently stable in aqueous media at neutral to basic pH.

The kinetics of imines formation and exchange is more rapid than in the acyl hydrazone case. However, the stability of the formed species is lower, and stabilising methods need to be undertaken. This can be done by reduction to the corresponding amines, e.g. by sodium cyanoborohydride.

A variety of hydrazide, amine and aldehyde building blocks were synthesised (Table 2-5), primarily chosen to contain various carbohydrate recognition groups in order to achieve an optimal binding to the target protein, Concanavalin A. In addition to using monofunctional building blocks, a set of difunctional entities were added as linker units to further probe the geometry of the binding sites. Building blocks with potentially low affinities were furthermore added for reference.

TABLE 2
Structures of the carbohydrate hydrazides H1-H4

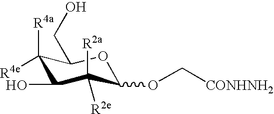

| Compound^{a} | α/β | R2a | R2e | R4a | R4e |
|---|---|---|---|---|---|
| H1 (Man) | α | OH | H | H | OH |
| H2 (Gal) | β | H | OH | OH | H |
| H3 (Glc) | β | H | OH | H | OH |
| H4 (GlcNAc) | β | H | NHAc | H | OH |

^{a}Man: D-Mannose;
Gal: D-Galactose;
Glc: D-Glucose;
GlcNAc: N-acetyl-D-Glucose

TABLE 3
Structures of the carbohydrate anilines AN1-AN5

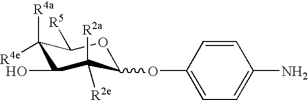

| Compound^{a} | α/β | R2a | R2e | R4a | R4e | R5 |
|---|---|---|---|---|---|---|
| AN1 (Man) | α | OH | H | H | OH | CH₂OH |
| AN2 (Gal) | β | H | OH | OH | H | CH₂OH |
| AN3 (Glc) | β | H | OH | H | OH | CH₂OH |
| AN4 (Ara) | β | H | OH | OH | H | H |
| AN5 (Xyl) | β | H | OH | H | OH | H |

^{a}Man: D-Mannose;
Gal: D-Galactose;
Glc: D-Glucose;
Ara: L-Arabinose;
Xyl: D-Xylose

TABLE 4
Structures of the carbohydrate amines AM1-AM5

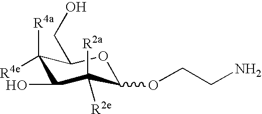

| Compound | α/β | R2a | R2e | R4a | R4e |
|---|---|---|---|---|---|
| AM1 (Man) | α | OH | H | H | OH |
| AM2 (Gal) | β | H | OH | OH | H |
| AM3 (Glc) | β | H | OH | H | OH |

^{a}Man: D-Mannose;
Gal: D-Galactose;
Glc: D-Glucose

TABLE 5
Structures of the carbohydrate aldehydes A1-A6

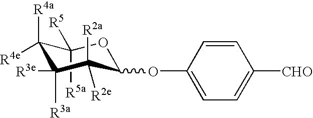

| Compound^{a} | α/β | R2a | R2e | R3a | R3e | R4a | R4e | R5a | R5e |
|---|---|---|---|---|---|---|---|---|---|
| A1 (Man) | α | OH | H | H | OH | H | OH | H | CH₂OH |
| A2 (Gal) | β | H | OH | H | OH | OH | H | H | CH₂OH |
| A3 (Glc) | β | H | OH | H | OH | H | OH | H | CH₂OH |
| A4 (GlcNAc) | β | H | OH | H | OH | H | OH | H | CH₂OH |
| A5 (GalNAc) | β | H | OH | H | OH | OH | H | H | CH₂OH |
| A6 (Fuc) | α | OH | H | OH | H | H | OH | CH₃ | H |

^{a}Man: D-Mannose;
Gal: D-Galactose;
Glc: D-Glucose;
GlcNAc: N-acetyl-D-Glucose;
GalNAc: N-acetyl-D-Galactose;
Fuc: L-Fucose

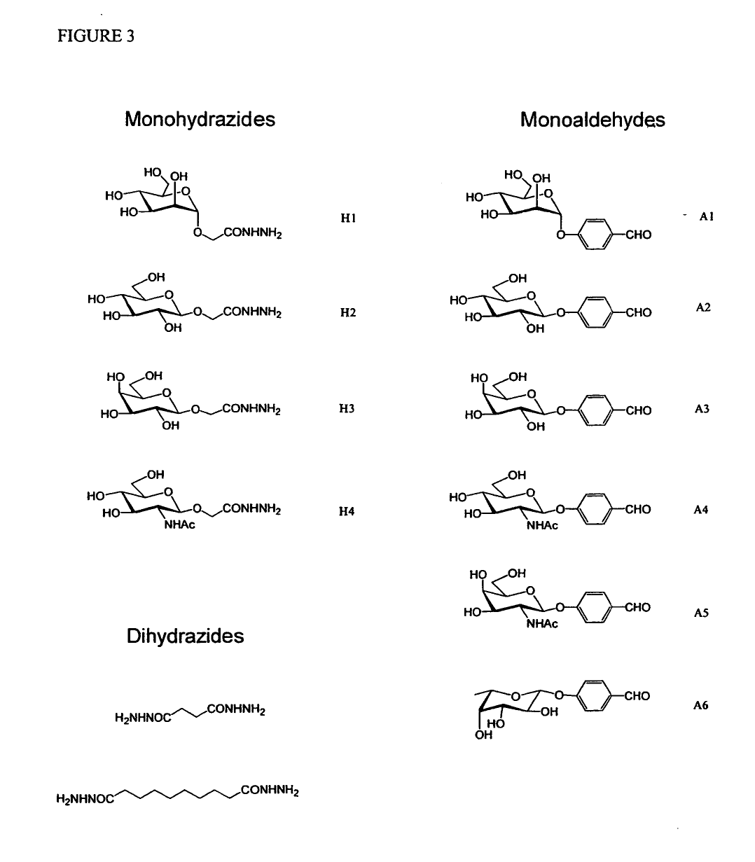

In one embodiment, the individual library components were subsequently used to produce acyl hydrazone DCLs containing either the hydrazide building blocks, the aldehyde building blocks, or both. Difunctional linker units of each type was also added, although not simultaneously in order to avoid polymerisation. An example of a mixed library is displayed in FIG. 3, composed of four monohydrazides, two dihydrazides, and five monoaldehyde units. By this combination of building blocks, a library composed of 50 different species is easily produced in one single operation.

Upon screening of these libraries against Concanavalin A, using a competitive binding assay, monoaldehyde A1 and sebacoyldihydrazide (SED) showed important contributions to the binding. These results indicate that the bis-mannoside (A1₂-SED) is likely to be the most active species in the library.

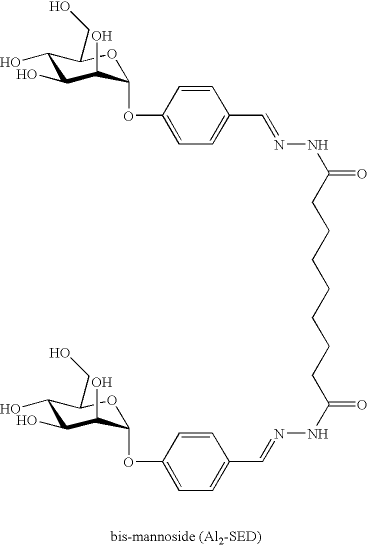

Synthesis of the library components of Example 1: The carbohydrate dimers were synthesised from the corresponding peracetylated 4-aminophenyl-glycosides, by condensation with the bis-dithiodiacids, followed by decetylation under standard Zemplén conditions (NaOMe/MeOH). The 4-aminophenyl derivates were all obtained from the commercial 4-nitrophenyl-glycosides following the same procedure.

4-aminophenyl-α-L-2,3,4-tri-O-acetyl-arabinopyranoside: The glycoside (500 mg) was slurried in dichloromethane (2.5 mL) and acetic anhydride (2.5 mL). A catalytic amount of dimethylaminophridine (DMAP) was added and the reaction allowed to proceed under argon at ambient temperature overnight. Following washing (water, HCl(aq.), NaHCO₃ (aq.), water), the product solution was concentrated and dried in vacuo. The product was subsequently dissolved in methanol (40 mL), palladium and charcoal was added (5%, 140 mg), and reduction was performed under H₂ for 4 hrs. After filtration of the catalyst, concentration and drying, the pure product was obtained as a white foam (93%). [α]_{D}²⁰=+29° (c=1,0 CWCl₃)¹³C NMR (50 MHz, CDCl₃, 298 K): δ=170.4, 170.2, 169.5, 118.8, 116.0, 100.6, 70.1, 69.2, 67.5, 63.1, 21.0, 20.8; FAB-MS⁺, 367.3 (M⁺).

4-aminophenyl-β-D-2,3,4-tri-O-acetyl-xylopyranoside: (94%) [α]_{D}²⁰=−27° ¹³C NMR (50 MHz, CD₃OD, 25°=171.7, 151.2, 144.5, 119.6, 117.7, 101.7, 73.3, 72.6, 70.4, 63.2, 20.8; 367.1 (M⁺).

4-aminophenyl-β-D-2,3,4,6-tetra-O-acetyl-galactopyranoside: (quant.) [α]_{D}²⁰=+4.60 (c=10.0 CHCl₃); ¹³C NMR (50 MHz, CDCl₃, 25° C.): δ=170.4, 170.2, 169.5, 150.0, 142.7, 119.0, 115.9, 101.2, 76.4, 71.0, 68.9, 67.1, 61.4, 20.7, 439.3 (M⁺); (S. Tahutake et al., Chem. Pharm. Bull. 1990(38), 13).

The other 4-aminophenyl derivatives had properties in agreement with the data reported in literature; 4-aminophenyl-β-D-2,3,4,6-tetra-O-acetyl-glucopyranoside, 4-aminophenyl-α-D-2,3,4,6-tetra-O-acetyl-mannopyranoside (D. Pagé et al., Bioorg. Med. Chem. 1996(4), 1949).

The carbohydrate dimers were obtained by the following procedure: A suspension of 4-aminophenyl-glycoside (0.57 mmol), 1-ethyl-3-(dimethylaminopropyl)-carbodiimide (0.63 mmol), and bis-dithiodiacid (0.29 mmol) was stirred for 4 hrs at room temperature under argon in dichloromethane (5 mL). Following washing (water, NaHCO₃ (aq.), water), and concentration of the organic phase, the product was purified by chromatography. Subsequent deprotection yielded the pure carbohydrate dimer.

Bis-mannoside 1: The intermediate product was purified by flash chromatography (SiO₂; dichloromethane/acetone 8:2, v/v), (67%), [α]_{D}²⁰=+94° (c=0.5, MeOH/H₂O 1:1, v/v); ¹³C NMR (50 MHz, CD₃OD, 25°); δ=172.0, 153.0, 133.0, 121.4, 116.7, 99.1, 73.9, 71.0, 70.6, 67.0, 61.3, 37.5, 34.7.24.8, ES-MS⁺, 745.5 (M+H).

Bis-galactoside 2 Purification by preparative TLC (Chromatotron, Al₂O₃, dichloromethane, 1% MeOH, v/v), (86%), [α]_{D}²⁰=−46° (c=0.2 in MeOH/H₂O 1:1, v/v); ¹³C NMR (50 MHz. CD₂OD/D₂O 1:1, 25° C.): δ=171.7, 154.3, 132.4, 122.5, 117.0, 101.5, 75.4, 72.9, 70.7, 68.6, 60.7, 35.7, 33.6; ES-MS+[calc. for C₃₀H₄₀N₂O₁₄S₂ 716.2 found] 739.3 (M+Na); [calc. C, 50.27; H, 5.62; N, 3.91: found C, 50.03; H, 5.66; N, 3.76].

Bis-galactoside 3: Purification by preparative TLC (Chromatotron, Al₂O₃, dichloromethane, 0.5% MeOH, v/v) (36%), [α]_{D}²⁰=43° (c=0.08 in MeOH/H₂O 1:1, v/v; (t, ¹³C NMR (50 MHz, CD₃OD/D₂O 1:1, 25° C.); δ=173.4, 154.2, 132.5, 122.6, 117.0, 101.5, 75.4, 72.9, 70.7, 68.6, 60.8, 37.4, 34.8, 24.8; ES-MS⁺ 767.2 (M+Na);

Bis-glucoside 4: Purification by flash chromatography (SiO₂; dichloromethane/acetone 8:2, v/v, (67%), [α]_{D}²⁰=−40° (c=0.3, MeOH/H₂O 1:1, v/v); ¹³C NMR (50 MHz, CD₃OD/D₂O 4:1, v/v. 25° C.): δ=171.1, 154.2, 132.8, 121.8, 116.9, 101.1, 76.5, 76.2, 73.4, 69.8, 61.0, 35.8, 33.7;

Bis-arabinoside 5: Purification by flash chromatography (SiO₂; dichloromethane/acetone 8:2, v/v), (42%), [α]_{D}²⁰=−6° (c=0.2, MeOH/H₂O 1:1, v/v); ¹³C NMR (50 MHz, CD₃OD/D₂O 1:1, 25° C.): δ=171.8, 154.1, 132.2, 122.6, 117.1, 101.5, 72.4, 70.6, 68.2, 66.2, 35.7, 33.6, ES-MS⁺ 657.0 (M+H).

Bis-xyloside 6: Purification by preparative TLC, (Chromatotron, Al₂O₃, dichloromethane/1% MeOH, v/v), (63%), [α]_{D}=−21° (c=0.3, MeOH/H₂O 1:1, v/v); ¹³NMR (50 MHz, CD₃OD/D₂O 1:1, 25° C.): δ=171.8, 153.9, 132.5, 122.6, 117.0, 101.4, 75.8, 73.1, 69.3, 65.3, 35.7, 33.6, ES-MS⁺ 679.2 (M+Na).

Generation and analysis of adaptive combinatorial libraries: To a solution of carbohydrate dimers 1-6 (10 μM, each) in buffer (900 μL, 100 mM Na-phosphate. 50 μM MnCl₂, pH 7.4), was added suspension of Con A-sepharose (50 μL, −5 nmol Con A), and dithiothreitol solution (50 μm, 20 mM). After equilibration at ambient temperature for two weeks, the suspension was filtered (Millipore. Amicon, YM-100), and the sepharose particles resuspended in buffer (400 μL). The bound carbohydrate dimers were released by addition of HCl (100 μL, 1 M), and thereafter renewed filtration, the solutions were analysed by RP-HPLC.

Generation and analysis of pre-equilibrated combinatorial libraries: The same protocol as for the adaptive libraries was used with the exception that Con A-Sepharose (50 μL) was added after the equilibration period. The resulting suspension was then allowed to further equilibrate during 1 hr at room temperature, and the bound species were eluted and analysed as previously mentioned.

Preparation of the Compounds of Example 2:

The carbohydrate hydrazides were readily synthesised in three steps from the corresponding peracetylated species. Thus, the glycosidic donor was coupled to methyl glycolate using either boron-trifluoride etherate or ferric chloride as promoters (Scheme 1). Simultaneous formation of the hydrazide and acetyl deprotection by hydrazine yielded the target compounds in excellent yields.

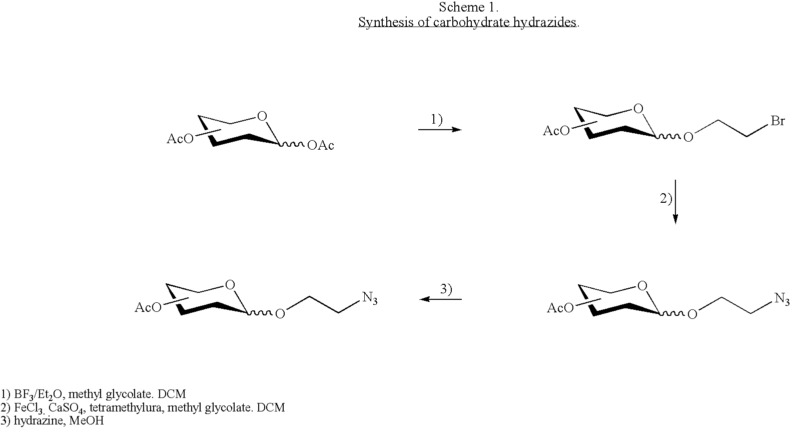

The aniline species were synthesised, as described, by reduction of the corresponding 4-nitrophenyl-glycosides using hydrogen and palladium on charcoal as catalyst. The primary amino functionality was introduced via the 2-bromo-ethylated carbohydrates (Scheme 2). The synthetic scheme comprised osidic coupling of 2-bromo-ethanol to the peracetylated glvcosidic donor using boron-trifluoride etherate as promoter, after which the bromide was substituted for an azide functionality. Reduction to amine under Lindlar conditions, followed by deprotection yielded the final carbohydrates in good yields.

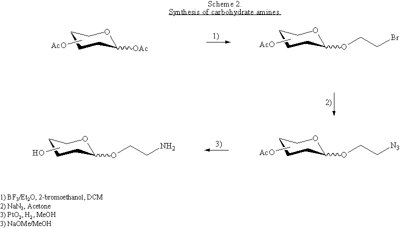

The carbohydrate aldehydes were prepared via the corresponding 1-chloro-, or 1-bromo-glycosides using either traditional Koenigs-Knorr or phase transfer catalysis protocols (Scheme 3). Thus, glycosyl halides were formed from the hydrogen halides in acetic acid/acetic anhydride in quantitative yields. Subsequent substitution with 4-hydroxybenzaldehyde using silver triflate as promoter, or under basic phase transfer conditions in water-dichloromethane yielded the peracetylated benzaldehydes. Final deprotection under Zemplen conditions resulted in the carbohydrate building blocks.

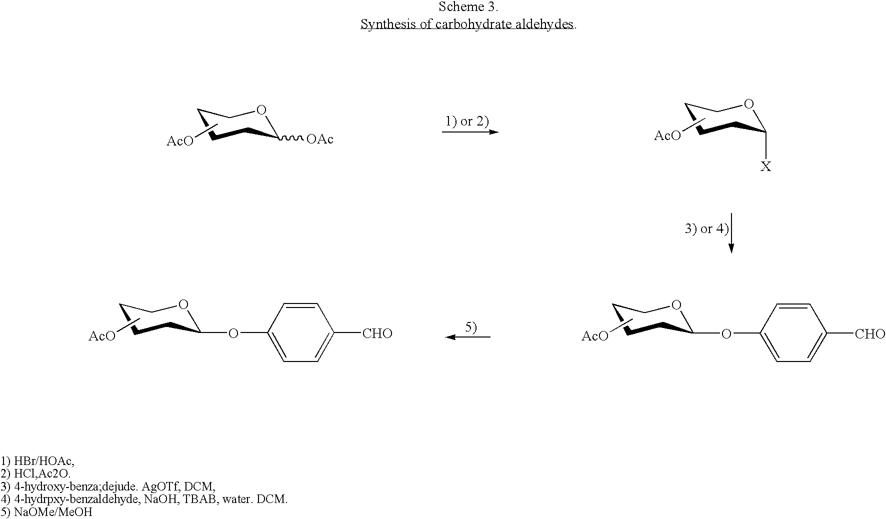

Preparation of Acylhydrazone Libraries:

In a typical example, stock solutions (25 mM) of all building blocks (monoaldehydes A1-A6, monohydrazides H1-H4, dihydrazides) were made in water, or water/methanol (1:1). The library was then made up of one part (40 μL) of each of these solutions, buffered with sodium acetate buffer (50 μL, 1 M, pH 4.0), and diluted with water (510 μL) to a final concentration of 1 mM of each building block, and 50 mM buffer. The resulting libraries were allowed to equilibrate for one week at room temperature to assure complete reaction, and used directly, or following concentration/dilution, in the subsequent screening tests.

Imine libraries can be prepared in essentially the same way, using amine and aldehyde building blocks, respectively. In this case most operations can be conducted at neutral to mildly basic conditions (pH 7-9). For sodium cyanoborohydride reduction, however, a pH below 6 is necessary. A excess of amine is here useful in order to overcome competing amino groups in the target protein, and to increase imine formation.

As is clear from the foregoing, the method according to the present invention can also be applied to the binding of carbohydrate substrates containing other residues than those used in the examples describe above, to lectins other than concanavalin and also to other carbohydrates binding proteins. As laid out above, the method according to the present invention can advantageously be applied to carbohydrates and carbohydrate receptors in particular sugar. However, the method according to the present invention lends itself to create dynamic combinatorial libraries of all kinds of ligands for targets binding at least two functionalities.

1. A method for selectively establishing a dynamic combinatorial library of ligands for a target which binds at least two functionalities, which method comprises the following steps:
(i) selecting a plurality of functionalities which upon combination with each other are capable of forming an entity which may bind to the at least two functionalities in the target;
(ii) selecting at least one spacer group to be located between the at least two functionalities, which spacer group is of an appropriate size and/or flexibility to allow the functionalities to fit into the binding sites on the target and allows a reversible bond formation and cleavage;
(iiia) creating discrete ligands by linking at least two identical or different functionalities by at least one spacer group; or
(iiib) linking the functionalities to fragments of the spacer group which contain functions allowing the said reversible bond formation under formation of the spacer group and cleavage of it;
(iv) mixing together a plurality of different discrete ligands and/or different functionalities;
(v) subjecting the mixture to conditions allowing a reversible bond formation and cleavage;
(vi) adding the target to the mixture;
(vii) identifying the functionality combinations which are most appropriate for the formation of a complex between the target and the active molecule.

2. A method for selectively establishing a dynamic combinatorial library of ligands for a target which binds at least two functionalities, which method comprises the following steps:
(I) selecting a plurality of functionalities which upon combination with each other are capable of forming an entity which may bind to the at least two functionalities in the target;
(II) selecting at least one spacer group to be located between the at least two functionalities, which spacer group is of an appropriate size and/or flexibility to allow the functionalities to fit into the binding sites on the target and allowing a reversible bond formation and cleavage;
(IIIA) creating discrete ligands by linking at least two identical or different functionalities by at least one spacer group; or
(IIIB) linking the functionalities to fragments of the spacer group which contain functions allowing the said reversible bond formation under formation of the spacer group and cleavage of it;
(IV) mixing together a plurality of different discrete ligands and/or different functionalities in the presence of the target;
(V) subjecting the mixture to conditions allowing a reversible bond formation and cleavage, hence a scrambling of the functionalities;
(VI) identifying the functionality combinations which are most appropriate for the formation of a complex between the target and the active molecule.

3. The method according to claim 1 or 2, wherein the final mixture is analysed and the result compared to the result obtained on a mixture obtained under identical conditions in the absence of the target.

4. The method according to any of the claims 1 to 3, wherein the said two or more functionalities are bound to one site in the target.

5. The method according to any of the claims 1 to 4, wherein the functionality is selected from the group consisting of heterocycles containing at least one atom selected from the group consisting of N, O, and S, amino acids, oligo- and polypeptides, carbohydrates and sugars and its derivatives and nucleic acid constituents and related groups.

6. The method according to any of the claims 1 to 5, wherein the functionality is a sugar, preferably a sugar selected from the group consisting of hexoses and pentoses.

7. The method according to any of the claims 1 to 6, wherein the spacer group contains functional groups from the group consisting of imines, hydrazones, acylhydrazones, semicarbazones and analogues thereof, acetals, esters, alkenes, alkines and disulphides, preferably from the group consisting of hydrazones, acylhydrazones, semicarbazones and analogues thereof, and disulphides.

8. The method according to claim 7, wherein the reversible bond cleavage and formation is attained by the addition of dithiotreitol, in the case of a disulphide containing spacer group.

9. The method according to any of the claims 1 to 8, wherein the target is a protein, an enzyme, a biological receptor or an antibody.

10. The method according to claim 9, wherein the said enzyme is a carbohydrate binding protein, preferably a lectin or a toxin, in particular an S-type lectin, a C-type lectin or a P-type lectin, or Shiga and Shiga-like toxins.

11. The method according to claim 9, wherein said ligand is selected from the group consisting of a substrate, an activator and an inhibitor of an enzyme, the target being an enzyme or an analogue thereof.

12. The method according to claim 9, wherein said ligand is selected from the group consisting of ligands, agonists and antagonists of a receptor.

13. The method according to claim 9, wherein said ligand is an antigen, said target being an antibody.

14. A dynamic combinatorial library which is obtainable by the method according to any of claims 1 to 14.
